(19) United States
(12) Patent Application Publication
(10) Pub. No.: US 2022/0257469 A1
(43) Pub. Date: Aug. 18, 2022

(54) PHARMACEUTICAL UNIT DOSE SYSTEMS FOR ORAL DRY SOLUTION AND SUSPENSION

(71) Applicant: Brillian Pharma Inc., Monmouth Junction, NJ (US)

(72) Inventor: Nuo Wang, Newtown, PA (US)

(73) Assignee: Brillian Pharma Inc., Monmouth Junction, NJ (US)

(21) Appl. No.: 17/618,033

(22) PCT Filed: Oct. 16, 2020
(86) PCT No.: PCT/US20/55962

§ 371 Date: Dec. 10, 2021

Related U.S. Application Data
(60) Provisional application No. 62/926,777, filed on Oct. 28, 2019.

(51) Int. Cl. A61J 1/20; A61J 1/18; A61J 1/14

(52) U.S. Cl. CPC A61J 1/20; A61J 1/1468; A61J 1/18

(57) ABSTRACT

This disclosure provides a pharmaceutical unit dose system. The unit dose system includes a unit-of-use self-dispersing dry composition for oral suspension. Also provided is a method for preparing a single unit dose of a dry pharmaceutical composition for oral administration. The unit dose system is advantageous in the convenience of use, dosing accuracy, long term storage, suspension uniformity over conventional multidose powder composition for oral suspension.

### CROSS-REFERENCE TO RELATED APPLICATIONS

This application claims priority under 35 U.S.C. § 119(e) to U.S. Provisional Patent Application No. 62/926,777, filed Oct. 28, 2019. The foregoing application is incorporated by reference herein in its entirety.

### FIELD OF THE INVENTION

This invention relates generally to pharmaceutical unit dose systems for oral administration and methods for preparing a single unit dose of a dry pharmaceutical composition for oral administration.

### BACKGROUND OF THE INVENTION

Oral liquids are traditional pharmaceutical dosage forms. They are most suitable for patients that have difficulty swallowing solid dosage forms such as tablets and capsules. Children younger than 7-8 years old predominately prefer oral liquids over oral solids (Mennella et al., Clin Ther. 2013; 35(8): 1225-1246; Mennella et al., BMC Pediatrics (2015) 15:130; Polaha et al., South Med J. 2008; 101(11):1106-12). Geriatric patients who are in old age or have suffered from certain illnesses, e.g., stroke or cancer, frequently have difficulty swallowing commonly prescribed oral solid dosage forms and, therefore, also prefer oral liquids for drug administration (Logrippo et al., Clinical Interventions in Aging 2017:12 241-251).

For drugs that are not stable in liquid forms, they are formulated into dry powder form (e.g., powder for suspension, dry suspension), then reconstituted into oral suspension before use. Oral powder for suspension is typically formulated into powder blends for reconstitution and packaged into multidose bottles. Before prescribing to users, the multidose bottles are usually reconstituted in a pharmacy by trained personnel. The reconstitution procedure involves measuring a predetermined volume of a medium (typically purified water), adding the measured medium into the multidose bottle, followed by vigorous shaking to thoroughly disperse the dry powder into the medium so that a uniform suspension can be obtained. The volume of the medium is defined in product labeling and needs to be accurately measured to provide an accurate concentration of the constituted suspension, so an accurate dose of each administration can be assured. The shaking needs to be sufficient to ensure complete reconstitution. The procedure sometimes takes multiple medium measurements/addition and shaking steps for certain drugs, e.g., REVATIO, FIRVANQ. Once reconstituted, the suspension is passed on to users to take home for self-administration. It has a very short shelf life, typically from a few days to less than 30 days. The suspension frequently needs to be stored under low temperatures (e.g., in refrigerator).

The current formulation and packaging forms of oral powder for suspensions, although having a long history of use and popular in pediatric and geriatric patient populations, are not without problems. The reconstitution procedure, apparently straight forward, can be prone to unsafe errors (Featured Articles, Life-threatening Errors with Flecainide Suspension in Children, Institute For Safe Medication Practices, April 2015; Multifactorial Causes of Tacrolimus Errors: Confusion with Strength/Formulation, Look-alike Names, Preparation Errors, and More; Institute For Safe Medication Practices, August 2017). Cases have been reported that unmixed antibiotics were dispensed before they were reconstituted (M. J. Gaunt, Pharmacy Times, November 2017). In such cases, the pharmacy involved had failed to reconstitute an antibiotic dry suspension before dispensing them to the patients or caregivers, resulting in pediatric patients being overdosed. Although such unfortunate events were caused by human error, they can also be attributed to both current pharmacy practice and the way the current reconstitution powder for suspension is packaged, dispensed, and used. Pharmacies know that it is best to reconstitute a dry powder for suspension with water right before it is picked up by parents for caregivers, so the suspension will be stable longer or will not be wasted if, for whatever reason, the prescription is not picked up. Thus, pharmacists generally reconstitute the powder only when they are sure the prescription is sure to be picked up and frequently when the parents or caregivers have already shown up and are waiting. In a busy pharmacy, the instruction to reconstitute before dispensing can be overlooked, causing the unreconstituted powder for suspension to be provided to patients. Also, the powder for suspension can be reconstituted with the incorrect amount of water in the pharmacy, e.g., more or less than the labeling specified amount/volume of water. As a result, the potency of the suspension is made higher or lower than the intended label claim dose. Patients can be overdosed or undertreated with the incorrectly constituted suspension.

To prevent these errors from happening, pharmacies will need to implement measures to their current practice, such as adding additional instructions, upgrade computer systems, incorporate an independent double-check/verification at the point of sale, etc. However, implementation of these measures will inevitably increase the waiting time and costs of a pharmacy operation.

Since most of the oral suspensions are prescribed in multidose bottles, a measuring device such as a measuring cup or spoon is usually provided alongside with the reconstituted suspension for users, typically untrained users, to measure a dose for each use. Accuracy of the measured dose can be affected by product resuspendability, viscosity, uniformity, and concentration/strength of the suspension. High viscosity suspensions cause more residue in the measuring device, and a rinse step needs to be added to assure complete delivery of the dose. Uniformity of a suspension can only be assured with sufficient agitation or sometimes vigorous shaking, which creates air bubbles in the suspension. Suspension with air bubbles trapped inside the liquid can cause the apparent measured volume to be lower than intended. Low dose suspensions, especially medications with a narrow therapeutic index (NPI) require more accurate volume measurement to provide intended dose. For example, Tacrolimus and levothyroxine require doses in micrograms. A small dose variation may cause extreme fluctuations in vivo. The impact of viscosity and uniformity of these suspensions on therapeutic outcome may be heightened when these inaccurate measuring devices are used. Syringes have been used to provide more accurate measurement for oral suspension. However, their use can cause live threatening drug misadministration, especially in hospital settings. It has been reported that intravenous (IV) syringes were used to measure oral liquids for oral administration and mistakenly connected to the intravenous line. Specially designed oral syringes that cannot be connected to standard IV lines or needles must be used. However, these syringes, once loaded with liquid medication, are often unlabeled and can easily cause drug misadministration.

Once reconstituted, a dry suspension is converted to a liquid form preparation. Storage conditions of a liquid oral suspension are critical to its stability, as the chemical stability, as well as drug potency and therapeutic effects of medications, can be affected by temperature, humidity, and light. Therefore, a constituted liquid suspension has a short so-called “in use” shelf-life that is affected by both lengths of time and storage conditions. Typical “in use” shelf-life usually ranges from 14 days to up to 60 days, which is a lot shorter than an average shelf-life of an un-opened medication (usually 24 months or longer). In most cases, the storage conditions for “in use” product is refrigeration at 2°-8° C. Studies have shown that users often do not follow labeling requirement to store their liquid oral suspensions under refrigeration. For many drugs, such as common antibiotics, their potency deteriorates quickly in liquid suspension and even faster if not refrigerated. Clavulanic acid, a drug used in combination with Amoxicillin for the treatment of bronchitis and urinary tract, skin, and soft tissue infections, can lose its potency by 30-40% in five days if not refrigerated, thus significantly undermining treatment outcomes.

Unit dose packaging can be a promising way of addressing the above-mentioned problems of oral liquids. Its current use, however, is limited to oral liquids and liquid oral suspensions. Its use is greatly limited due to drug stability in a dissolved state. There are a vast number of medications that are not sufficiently stable in liquid form for a commercially viable shelf life. Therefore, powder for suspension in unit dose package is needed, and such need is currently largely unmet, for several reasons: (1) the unit dose powder for suspension would need to be packaged in small volume containers (e.g., 10 to 30 ml) and sealed. Once the seal is opened, reconstitution medium (usually water) is added to resuspend the powder into suspension. Due to small volume of the package container, vigorous shaking can not be used to prevent spillage and medication loss. But most of current powder for suspensions require sufficient mixing by vigorous shaking to assure proper suspension, uniformity, and complete dosing of the medication; (2) the unit dose package of powder for suspension requires accurately weighed small amount of powder (mostly less than 500 mg per dose) be filled in a small container. Dry powder is difficult to fill with sufficient and consistent accuracy. It is difficult to control the weight variation to meet the regulatory requirements (usually within 5-7% relative standard deviation); (3) Many dry powder suspensions contain fine particle materials. Like dust, they float in the air and may settle on the rim of package container and may adversely affect the sealing integrity. Often the fine particle materials are micronized actives. The floating dust of micronized actives can be harmful to handler, especially when the actives are cytotoxic oncology drugs. Although the dust can be minimized by granulating the powder blend or by making the blend more granular, it may require longer dispersion time at reconstitution.

Therefore, there is an unmet need for pharmaceutical unit dose systems for oral administration and methods for preparing a single unit dose of a dry pharmaceutical composition for oral administration.

### SUMMARY OF THE INVENTION

This disclosure addresses the need mentioned above in a number of aspects. In one aspect, the unit dose system comprises (i) a self-dispersible dry pharmaceutical composition for oral administration, wherein the pharmaceutical composition comprises at least one active agent in the amount of a single administrative dose and at least one pharmaceutically acceptable excipient that allows the dry pharmaceutical composition to self-disperse in the presence of a liquid medium; (ii) a container for packaging and reconstituting the dry pharmaceutical composition, wherein the container has a volume at least five-fold greater than the volume of the dry pharmaceutical composition; and (iii) a seal cover removably secured to the container by a sealing contact with an opening of the container.

In another aspect, this disclosure also provides a method for preparing a single unit dose of a dry pharmaceutical composition for oral administration. The method comprises (a) providing the pharmaceutical unit dose system as described above; (b) detaching the seal cover, partially or completely, from the opening of the container; and (c) adding a reconstitution liquid medium into the container to approximately the volume marking and allowing the dry pharmaceutical composition to self-disperse to form an oral solution or suspension.

In another aspect, this disclosure further provides a method for forming the pharmaceutical unit dose system for oral administration as described above. The method comprises: (a) forming the container with an opening; (b) preparing the self-dispersing dry pharmaceutical composition; (c) packaging a single dose of the dry pharmaceutical composition into the container; and (d) securing the seal cover to the container by a sealing contact over the opening of the container.

Alternatively, the method for forming the pharmaceutical unit dose system for oral administration comprises: (a) forming the container with an opening; (b) preparing a solution or a liquid suspension of the self-dispersing dry pharmaceutical composition; (c) adding the solution or the liquid suspension of a single dose of the pharmaceutical composition into the container; (d) freeze-drying the solution or the liquid suspension to form a freeze-dried cake of the pharmaceutical composition; and (e) securing the seal cover to the container by a sealing contact over the opening of the container.

In yet another aspect, this disclosure additionally provides a kit for preparing a single unit dose of a dry pharmaceutical composition for oral administration. The kit comprises (i) the unit dose system as described above; (ii) a second container comprising an aqueous carrier; and (iii) optionally an instruction for reconstituting the pharmaceutical composition using an aqueous carrier. In some embodiments, the aqueous carrier comprises water, e.g., purified water, flavored water, or an isotonic solution.

In some embodiments, the pharmaceutical composition further comprises a preservative, a coloring agent, an antioxidant, a pH adjusting agent, an anti-foaming agent, a suspending agent, a thickening agent, and a combination thereof.

In some embodiments, the excipient comprises a highly water-soluble material, a disintegrant, or a combination thereof. In some embodiments, the excipient comprises a taste-masking agent. In some embodiments, the excipient comprises a matrix-forming agent.

In some embodiments, the dry pharmaceutical composition is in the form of a powder blend. The powder blend may include formulated granules produced from a dry or wet granulation process.

In some embodiments, the dry pharmaceutical composition is a freeze-dried pharmaceutical composition. The freeze-dried pharmaceutical composition can be prepared by in situ freeze-drying a liquid suspension or solution of the pharmaceutical composition in the container. In some embodiments, the dry pharmaceutical composition is in the form of a freeze-dried cake.

In some embodiments, the dry pharmaceutical composition comprises an ion-exchange resin that complex with the active agent to conceal the taste of the active agent. The ion-exchange resin may be micronized. In some embodiments, the ion-exchange resin has a particle size of less than 50 μm. In some embodiments, the ion-exchange resin is Amberlite IRP-69, Amberlite IRP-64, Colestipol hydrochloride, or Duolite AP143/1093.

In some embodiments, the active agent is selected from the group consisting of analeptic agents; analgesic agents; anesthetic agents; antiasthmatic agents; antiarthritic agents; anti-cancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyper activity disorder active agents; cardiovascular agents including calcium channel blockers and antianginal agents; central nervous system (CNS) agents; beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide active agents; psychoStimulants; sedatives; Sialagogues, steroids; Smoking cessation agents; Sympathomimetics; tranquilizers; vasodilators; beta-agonist; tocolytic agents; and combinations thereof. In some embodiments, the active agent can be a cytotoxic anti-cancer or anti-tumor agent, an antiviral, or an anti-bacterial agent.

In some embodiments, the liquid medium is water (e.g., purified water, sweetened water, or flavored water) or an isotonic solution.

In some embodiments, the container has a dual function as a primary package component and a reconstitution device for the pharmaceutical composition. In some embodiments, the container is cylindrical or substantially cylindrical. The container may include a volume marking indicating a volume for reconstitution of the pharmaceutical composition. In some embodiments, the volume for reconstitution of the pharmaceutical composition indicated by the volume marking is about or less than 80% of the interior volume defined by the container. In some embodiments, the interior volume of the container is between about 5 mL and about 50 mL (e.g., between about 10 mL and about 40 mL, between about 10 mL and about 30 mL).

In some embodiments, the container is formed of a plastic selected from the group consisting of polyethylene (PE) including high-density polyethylene (HDPE) and low-density polyethylene (LDPE), polyvinyl chloride (PVC), polypropylene (PP), nylon, polyterpthalate (PET), polycarbonate, cyclic olefin copolymers (COC), polystyrene, and a combination thereof. In some embodiments, the container is formed of glass.

In some embodiments, the container is a rigid body container. In some embodiments, the container comprises an inner protrusion allowing the pharmaceutical composition to be held in place. The inner protrusion may be a circumferential protrusion. In some embodiments, the inner protrusion is part of a recessed ring positioned above the bottom of the container.

In some embodiments, the seal cover comprises product information. The product information may include a product name, dosage, ingredients, lot number, expiration date, company information, contact information, serialization barcode, warning message, instruction for use, or a combination thereof.

In some embodiments, the seal cover is formed of aluminum, paper, plastic, or a combination thereof. The seal cover can be an aluminum foil or a plastic film. In some embodiments, the seal contact to secure the seal cover to the container is provided by an adhesive.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A and 1B (collectively “FIG. 1”) show cross-section views of an example of the pharmaceutical unit dose system. FIG. 1A shows a cross-section view of an example of the pharmaceutical unit dose system containing a unit-of-use self-dispersible dry pharmaceutical composition. FIG. 1B shows a cross-section view of an example of the pharmaceutical unit dose system, in which the container includes an inner protrusion that helps to hold a free-dried cake of the pharmaceutical composition in place at the lower section of the container. 1: Container wall; 2: Peel-open lidding; 3: Unit-of-use self-dispersible dry pharmaceutical composition; 4: Volume-estimation marking; and 5: Protrusion holding freeze-dried cake in place.

FIG. 2 shows a see-through 3D view of an example of the pharmaceutical unit dose system. 1: Printed area; 2: Peel-open lidding; 3: Volume-estimation marking; 4: Unit-of-use self-dispersible dry pharmaceutical composition.

FIG. 3 shows a see-through 3D view of an example of the pharmaceutical unit dose system, in which the container includes an inner protrusion that helps to hold a free-dried cake of the pharmaceutical composition in place at the lower section of the container. 1: Printed area; 2: Peel-open lidding; 3: Volume-estimate marking; 4: Freeze-dried cake hold-in-place feature; 5: Unit-of-use self-dispersible dry pharmaceutical composition.

FIG. 4 shows an exemplary process for preparing a single unit dose of a dry pharmaceutical composition for oral administration. 1: Peel open & remove the lidding; 2: Add a medium (e.g., water) to approximately the volume-estimation mark to form a solution/suspension; 3: Ingest per oral route (e.g., drink directly out of the container).

FIGS. 5A and 5B (collectively “FIG. 5”) show unit-of-use, self-dispersing dry suspension of Bexarotene before reconstitution (FIG. 5A) and after 30 seconds of the elapsed time of reconstitution (FIG. 5B).

FIGS. 6A and 6B (collectively “FIG. 6”) show unit-of-use, self-dispersing dry suspension of Abiraterone Acetate before reconstitution (FIG. 6A) and after 30 seconds of the elapsed time of reconstitution (FIG. 6B).

FIGS. 7A and 7B (collectively “FIG. 7”) show unit-of-use, self-dispersing dry suspension of Acyclovir before reconstitution (FIG. 7A) and after 30 seconds of the elapsed time of reconstitution (FIG. 7B).

FIGS. 8A and 8B (collectively “FIG. 8”) show unit-of-use, self-dispersing dry suspension of Felodipine before reconstitution (FIG. 8A) and after 30 seconds of the elapsed time of reconstitution (FIG. 8B).

FIGS. 9A and 9B (collectively “FIG. 9”) show unit-of-use, self-dispersing dry suspension of Guanfacine HCl produced in a plastic container, before reconstitution (FIG. 9A) and after 30 seconds of the elapsed time of reconstitution (FIG. 9B; reconstituted content was transferred to a glass container for visual observation).

FIGS. 10A and 10B (collectively “FIG. 10”) show unit-of-use, self-dispersing dry suspension of Nifedipine before reconstitution (FIG. 10A) and after 30 seconds of the elapsed time of reconstitution (FIG. 10B).

FIGS. 11A and 11B (collectively “FIG. 11”) show unit-of-use, self-dispersing dry suspension of Oseltamivir phosphate produced in a plastic container, before reconstitution (FIG. 11A) and after 30 seconds of the elapsed time of reconstitution (FIG. 11B; reconstituted content was transferred to a glass container for visual observation).

FIGS. 12A and 12B (collectively “FIG. 12”) show unit-of-use, self-dispersing dry solution of Amlodipine Besylate before reconstitution (FIG. 12A) and after 30 seconds of the elapsed time of reconstitution (FIG. 12B).

### DETAILED DESCRIPTION OF THE INVENTION

This disclosure, in one aspect, provides a pharmaceutical unit dose system for oral administration. The unit dose system as disclosed has at least the following advantages: (1) It does not need to be prepared by a trained professional, e.g., pharmacist, and does not need to be reconstituted in a pharmacy. Instead, it can be reconstituted in any settings, such as home, office, school, or the like, or even on-the-go or traveling environments. The medium for reconstitution does not require accurate volume measurement, and the potency of the product would be not compromised. The preparation will be simple, and no vigorous shaking is required. Uniformity of the product is not compromised in the absence of vigorous shaking; (2) It is in unit-of-use packaging, thus dose division is not required; (3) It has a long shelf-life as it is stored in a dry state; and it does not need an “in use” shelf-life as the reconstitution only happens at the time of use and therefore requires no refrigeration for the “in-use” storage as required for most traditional reconstituted oral liquids; (4) It is easy for carrying and suitable for travelers. It can be carried around without the need for a cool bag or ice pack. Reconstitution is simple and does not require a separate dosing device; and (5) It has no or minimal presence of floating powder.

It discloses a unit-of-use self-dispersible dry pharmaceutical composition for oral administration that includes at least one active ingredient in the amount of a single administrative dose, and at least one pharmaceutically acceptable excipient enabling the self-dispersing feature of the dry pharmaceutical composition. The self-dispersible dry pharmaceutical composition is housed and sealed in a container (e.g., a rigid bodied container) with a removable seal (e.g., a peelable seal) to preserve the stability/integrity of the active(s) and the self-dispersing feature of the dry pharmaceutical composition. The peelable seal and/or label on the container may include necessary unit packaged product information required by a regulatory agency, such as product name, strength, lot number, expiration date, company information, and serialization barcode.

The container of the disclosed unit dose system possesses a dual function as a primary package compartment and a reconstitution device for the pharmaceutical composition. At the time of drug administration, the seal can be peeled open, a reconstitution liquid, such as water, is added into the container to approximately the volume marking and disperse the dry pharmaceutical composition into an oral solution or suspension without the need for vigorous shaking. The single dose of the constituted solution or suspension can be ingested directly out of the container. There is no dose division and a separate reconstitution container/device needed.

This disclosure additionally provides a kit for preparing a single unit dose of a dry pharmaceutical composition for oral administration. The kit comprises (i) the unit dose system as described above; (ii) a second container comprising an aqueous carrier; and (iii) optionally an instruction for reconstituting the pharmaceutical composition using aqueous carrier. The aqueous carrier can be water, e.g., purified water, flavored water, or a solution.

### A. PHARMACEUTICAL UNIT DOSE SYSTEM AND METHODS FOR PREPARING A SINGLE UNIT DOSE OF A DRY PHARMACEUTICAL COMPOSITION

The unit dose system may include a unit-of-use, self-dispersible dry pharmaceutical dry composition packaged in a small volume unit-of-use container. The unit-of-use, self-dispersible dry pharmaceutical composition may include at least one therapeutically active pharmaceutical ingredient and at least one pharmaceutically acceptable excipient that enables the composition to self-disperse in an aqueous medium to form a liquid suspension in a small volume unit-of-use container.

In one aspect, the unit dose system comprises: (i) a self-dispersible dry pharmaceutical composition for oral administration. The pharmaceutical composition comprises at least one active agent in the amount of a single administrative dose and at least one pharmaceutically acceptable excipient that allows the dry pharmaceutical composition to self-disperse in the presence of a liquid medium; (ii) a container for packaging and reconstituting the dry pharmaceutical composition, wherein the container has a volume at least five-fold greater than the volume of the pharmaceutical composition; and (iii) a seal cover removably secured to the container by a sealing contact with an opening of the container.

In another aspect, this disclosure also provides a method for preparing a single unit dose of a dry pharmaceutical composition for oral administration. The method comprises: (a) providing the pharmaceutical unit dose system as described above; (b) detaching the seal cover, partially or completely, from the opening of the container; and (c) adding a reconstitution liquid medium into the container to approximately the volume marking and allowing the dry pharmaceutical composition to self-disperse to form an oral solution or suspension.

(a) Pharmaceutical Compositions

In some embodiments, the unit-of-use, self-dispersible dry pharmaceutical composition includes at least one pharmaceutically acceptable excipient that serves as a taste-masking agent that either masks or conceals unpleasant taste of the pharmaceutically active ingredient(s). The pharmaceutical excipient that serves as a taste-masking agent can be an ion-exchange resin that complexes with the pharmaceutically active ingredient(s). In some embodiments, the unit-of-use, self-dispersible dry pharmaceutical composition can further include preservatives, coloring agents, antioxidants, pH adjusting agents, anti-foaming agents, suspending agents, and thickening agents.

The active pharmaceutical ingredient can be any compounds or mixture of compounds that are intended to furnish pharmacological activity or other direct effects in the diagnosis, cure, mitigation, treatment, or prevention of disease, or to affect the structure or any function of the body of humans or animals through oral route of administration. The active pharmaceutical ingredient can be from both synthesized and natural sources.

In some embodiments, the active agent is selected from the group consisting of analeptic agents; analgesic agents; anesthetic agents; antiasthmatic agents; antiarthritic agents; anti-cancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyperactivity disorder active agents; cardiovascular agents including calcium channel blockers and antianginal agents; central nervous system (CNS) agents; beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide active agents; psychoStimulants; sedatives; Sialagogues, steroids; Smoking cessation agents; Sympathomimetics; tranquilizers; vasodilators; beta-agonist; tocolytic agents; and combinations thereof.

In some embodiments, the active agent can be a cytotoxic anti-cancer or anti-tumor agent, an antiviral, or an anti-bacterial agent.

The pharmaceutical excipients that enable the unit-of-use, self-dispersible dry pharmaceutical composition to be self-disperse in an aqueous medium can be surfactants, highly water-soluble materials, and disintegrants. They can be used individually or in combination. They enable or facilitate the unit-of-use, self-dispersible dry pharmaceutical composition to disperse into a suspension or dissolve into a solution in a small volume unit-of-use container in a short period of time, i.e., in less than 60 seconds or less than 30 seconds, without the need of vigorous shaking. The small volume unit-of-use container has a volume at least five times greater than the volume of the dry pharmaceutical composition. The volume of the container can be less than 50 mL, e.g., less than 30 mL.

Surfactants are amphiphilic compounds that lower the interfacial tension between two liquids, between a gas and a liquid, or between a liquid and a solid. When the unit-of-use, self-dispersible dry pharmaceutical composition is in contact with an aqueous medium such as water, surfactants act as wetting agents that help the composition to be wetted and promote faster dissolving or dispersion of the composition. The surfactants can also act as emulsifiers to disperse water-insoluble compositions to be dispersed as an emulsion. Surfactants may include ionic surfactants and nonionic surfactants.

The ionic surfactants can be anionic surfactants, including without limitation ammonium lauryl sulfate, sodium lauryl sulfate, dioctyl sodium sulfosuccinate, perfluoro octane sulfonate, perfluorobutane sulfonate, alkyl-aryl ether phosphates, alkyl ether phosphates, and sodium stearate; cationic surfactants such as octenidine dihydrochloride, cetrimonium bromide (CTAB), cetylpyridinium chloride (CPC), benzalkonium chloride (BAC), benzethonium chloride (BZT), dimethyldioctadecylammonium chloride, and dioctadecyldimethyl ammonium bromide (DODAB), and zwitterionic surfactants such as phospholipids.

Phospholipids are lipids that can form lipid bilayers because of their amphiphilic properties. The structure of the phospholipid molecule generally consists of two hydrophobic fatty acid “tails” and a hydrophilic “head” consisting of a phosphate group. The two components are typically linked together by a glycerol moiety. The phosphate groups can be in acid form or modified with choline, ethanolamine, or serine through phosphodiester linkage.

The nonionic surfactants may include without limitation ethoxylates, fatty alcohol ethoxylates, alkylphenol ethoxylates, fatty acid ethoxylates, ethoxylated amines, and/or fatty acid amides, terminally blocked ethoxylates, fatty acid esters of polyhydroxy compounds, fatty acid esters of glycerols, and fatty acid esters of sorbitols (Spans & Tweens).

Highly water-soluble materials are highly hydrophilic and have high solubility in water. These materials include monosaccharide and disaccharide sugars, salts, amino acids, and hydrophilic polymers. Examples of monosaccharide and disaccharide sugars include without limitation glucose, fructose, galactitol, mannitol, lactose, sucrose, xylose, ribose, mannose, dextrose, maltose, trehalose, sorbitol, xylitol, maltose, glycerol, erythritol, threitol, arabitol, xylitol, ribitol, fucitol, iditol, inositol, volemitol, isomalt, maltitol, lactitol, maltotriitol, maltotetraitol, polyglycitol, and the combination thereof.

Highly water-soluble salts may include without limitation sodium, potassium salts such as sodium chloride, potassium chloride, and the combination thereof.

Highly water-soluble amino acids are charged or polar amino acids. Examples of charged or polar amino acids include arginine, lysine, aspartic acid, glutamic acid, histidine, serine, threonine, cysteine, and the combination thereof.

Hydrophilic polymers can be natural, modified natural, and synthetic polymers. Examples of hydrophilic polymers may include without limitation gelatin, Arabic gum, sodium alginate, acacia gum, agarose, xanthan gum, carrageenan, pectin, polyvinyl pyrrolidone, hydroxypropyl methylcellulose, hydroxypropyl cellulose, sodium carboxymethyl cellulose, polyacrylic acids, polyethylene glycols, polyethylene oxides, and the combination thereof.

The disintegrants can be water-insoluble but highly hydrophilic and swellable materials. They help break dry powder compositions such as granules, agglomerates, tablets into primary particles. Examples of disintegrants include cross-linked or non-crosslinked synthetic or natural polymers, such as sodium starch glycolate, cross-linked polyvinylpyrrolidone, croscarmello se sodium, and alginic acid.

These highly water-soluble materials can be used individually or in combination to obtain the best self-dispersing effect. They are uniformity dispersed in the unit-of-use, self-dispersible dry pharmaceutical composition. To obtain a better self-dispersing effect, these materials can be distributed between primary particles that to be dispersed after reconstitution.

The unit-of-use, self-dispersible dry pharmaceutical composition can be in different forms and produced by various methods.

In one embodiment, the unit-of-use self-dispersible dry pharmaceutical composition can be a powder blend. The powder blend can be produced by directly mixing an active with an excipient that enables self-dispersing behavior. In another embodiment, the unit-of-use self-dispersible dry pharmaceutical composition can be a powder blend contains formulated granules. The granules can be produced by conventional dry or wet granulation processes, in which an active is mixed with excipients and granulated. The granules will break apart quickly and self-disperse into a suspension or dissolve into a solution when in contact with a liquid medium.

In some embodiments, the unit-of-use self-dispersing dry pharmaceutical composition can be produced by a freeze-drying process, e.g., directly freeze-dried in a proper-sized container that can serve as both packaging and reconstitution device. In such a case, an aliquot of the solution or suspension that contains a unit-of-use dose of active can be filled into the containers. The filled containers can be placed into a freeze dryer, and the filled solution or suspension can be dried into a powder form or a cake form inside the container.

When the active is dusting, potent, and/or relatively toxic, the freeze-dried cake form is desirable to avoid potentially harmful effects during reconstitution. Alternatively, the bulk solution or suspension can be spread onto a tray for bulk drying. The tray is placed in a freeze-dryer and dried into bulk cakes. The freeze-dried bulk cakes can then be broken into powder or granules by proper milling or screen step to form a flowable blend with or without additional excipients. An aliquot of the flowable blend containing a single therapeutic dose can be filled into the unit-of-use container.

The filling of the unit-of-use composition in liquid forms provides better single use dose accuracy for unit-of-use containers than filling in powder/blend form. This is particularly beneficial for potent low dose actives and narrow therapeutic index (NPI) actives. The filling in liquid forms is beneficial in managing containment of potent and toxic actives (e.g., cytotoxic oncology drugs) by eliminating dusting during the filling process.

In some embodiments, the unit-of-use self-dispersible dry pharmaceutical composition contains an excipient that masks or conceals the unpalatable taste of an active pharmaceutical ingredient. The taste-masking excipient can be a flavoring agent that can be one of natural or synthetic flavoring agents, such as bananas flavor, grape flavor, cherry, pineapple, raspberry, vanillin flavor, coconut, cinnamon, strawberry, lime, peach, orange, lemon, chocolate flavors, caraway, clove, dill, orange, peppermint, or combination thereof. The taste-masking excipient can also be a sweetener, selected from natural or synthetic sweeteners, such as Glucose, Fructose, Galactose, Sucrose, Lactose (milk sugar), Maltose, Agave, Fructose, High-fructose Corn Syrup (HFCS), Honey, saccharin, aspartame, acesulfame potassium (Ace-K), sucralose, neotame, and advantame, and Stevia, or combination thereof.

In some embodiments, the unit-of-use self-dispersible dry pharmaceutical composition may include an ion-exchange resin that complexes with the active to conceal its unpleasant taste. The suitable ion exchange resins include anionic ion-exchange resins or cationic ion-exchange resins. These resins can be a cross-linked sulfonated polystyrene ion-exchange resin, a cross-linked methacrylic acid, and divinylbenzene copolymer ion-exchange resin, a cross-linked copolymer of diethylenetriamine and 1-chloro-2, 3-epoxy propane ion-exchange resin, or a cross-linked copolymer of styrene and divinylbenzene with quaternary ammonium functionality ion-exchange resin such as commercially available Amberlite IRP-69, Amberlite IRP-64, Colestipol hydrochloride, or Duolite AP143/1093, or combination thereof. The drug-ion exchange resin complex can be dispersed in a suitable medium to form a suspension. The suspension can be freeze-dried in the container with other excipients that provide the self-dispersing feature. The drug-ion exchange resin complex can also be in micronized form to improve both taste and mouthfeel of the constituted suspension. For example, the micronized drug-ion exchange resin complex may have an average particle size of about or less than 50 μm.

In some embodiments, matrix-forming excipients can be added to the solution or suspension before subjecting to the freeze-drying process, so that when the freeze-dried cake in the container will maintain its cake shape after freeze-drying and will not crumble in the container before being constituted.

The unit-of-use self-dispersible dry pharmaceutical composition can further include additional additives that enable the invention to function safely and effectively. Additional inactive ingredients, such as preservatives, coloring agents, antioxidants, flavoring agents, sweetener, anti-foaming agents, and pH adjusting agents, can be added to the solution or suspension.

In some embodiments, the dry pharmaceutical composition may include a pH adjusting agent. The pH adjusting agents can be acidic or basic compounds such as acetic acid, adipic acid, ammonium aluminum sulphate, ammonium, bicarbonate, ammonium carbonate, ammonium citrate, dibasic, ammonium citrate, monobasic, ammonium hydroxide, ammonium phosphate, dibasic, ammonium phosphate, monobasic, calcium acetate, calcium acid pyrophosphate, calcium carbonate, calcium chloride, calcium citrate, calcium fumarate, calcium gluconate, calcium hydroxide, calcium lactate, calcium oxide, calcium phosphate, dibasic, calcium phosphate, monobasic, calcium phosphate, tribasic, calcium sulphate, citric acid, fumaric acid, gluconic acid, hydrochloric acid, lactic acid, magnesium carbonate, magnesium citrate, magnesium fumarate, magnesium hydroxide, magnesium oxide, magnesium phosphate, magnesium sulphate, malic acid, phosphoric acid, potassium acid tartrate, potassium aluminum sulphate, potassium bicarbonate, potassium carbonate, potassium chloride, potassium citrate, potassium fumarate, potassium hydroxide, potassium lactate, potassium phosphate, dibasic, potassium phosphate, tribasic, potassium sulphate, sodium acetate, sodium bicarbonate, sodium bisulphate, sodium carbonate, sodium citrate, sodium hydroxide, sodium phosphate, dibasic, sodium phosphate, monobasic, sulphuric acid, tartaric acid, or combination thereof.

In some embodiments, the dry pharmaceutical composition can further include an antioxidant for enhancing drug stability. The antioxidants can be butylated hydroxyanisole, butylated hydroxytoluene, tert-butylhydroquinone, propyl gallate, erythorbic acid, ascorbyl palmitate, tocopherols, ethoxyquin, phosphates, ethylene diamine tetraacetic acid, tartaric acid, citric acid, lecithin, ascorbic acid, sulfites (as sulfur dioxide), ascorbyl stearate, or combination thereof.

In some embodiments, the dry pharmaceutical composition can include a suspending or thickening agent to modulate the viscosity of the constituted dry pharmaceutical composition. Examples of suspending or thickening agents may include alginates, methylcellulose, hydroxyethylcellulose, carboxymethylcellulose, sodium carboxymethylcellulose, microcrystalline cellulose, acacia, tragacanth, xanthan gum, bentonite, carbomer, carrageenan, powdered cellulose, or gelatin, or combinations thereof.

In some embodiments, the dry pharmaceutical composition can include a preservative selected from amino aryl acid esters such as methylparaben ethylparaben propylparaben butylparaben, alkyl/aryl alcohols such as benzyl alcohol, chlorobutanol, phenols such as phenol, meta cresol, chlorocresol, alkyl/aryl acids such as benzoic acid sorbic acid, organic mercurial such as thiomersal, phenylmercuric nitrate, diols such as bronopol, propylene glycol, quaternary ammonium compounds such as benzalkonium chloride, benzethonium chloride, or combination thereof.

In some embodiments, the dry pharmaceutical composition can also include a coloring agent selected from organic dyes or their lakes, natural colors, or Inorganic colors or mineral colors. Dyes are synthetic, chemical compounds that exhibit certain colors. Examples include Tartrazine, Erythrosine, Sunset Yellow, and Patent Blue V. Lakes are Aluminum salts of FD&C water-soluble dyes extended on a substratum of alumina. FD&C lakes are largely water-insoluble forms of the common synthetic water-soluble dyes and are available in six basic colors: one yellow, one orange, two reds (a pink-red and an orange-red), two blues (a green-blue and a royal blue). They can be blended to create more lake colors as needed, including brown, green, orange, red, yellow, and purple. Examples include aluminum lakes, brilliant blue lake, sunset yellow lake, amaranth lake, Allura red lake, indigo carmine lake, quinoline yellow lake. Examples of natural colors or vegetable and animal colors include caramel, cochineal (a dried insect), and carmine (the aluminum lake of the coloring matter of cochineal), riboflavin and anthocyanins, paprika oleoresin, beetroot red, annatto, and curcumin (turmeric). Examples of inorganic or mineral colors include red and yellow ferric oxides, titanium dioxide.

The containers containing the freeze-dried powder or cake can then be sealed with the peel-open lidding. Alternatively, the solution or suspension can be spread onto trays that can be dried into cakes in a freeze dryer. The freeze-dried cakes can then be broken into powder or granules by proper milling step. The powder or granules that contains a single therapeutic dose can be filled into the container and sealed with the peel-open lidding. The peel-open lidding may also possess a child resistant feature. Further, a cap with child resistant feature can be applied over the peel-open lidding.

In some embodiments, a dry pharmaceutical composition is reconstituted in an appropriate liquid, so that a solution, suspension, emulsion, Of dispersion is formed. Dry powder formulations may be reconstituted with a liquid carrier so as to generate a resulting reconstitute composition. In some embodiments, the liquid carrier can be an aqueous carrier. An appropriate liquid carrier for reconstitution of dry powder compositions may comprise an aqueous carrier such as water (e.g., sterile water, purified water, water for injection, flavored water) or an isotonic solution.

Aqueous carriers are known in the art, and include, but are not limited to, purified water, sterile water, water for injection, or an isotonic solution (when the dry powder composition does not contain an ion-exchange resin-active complex). An isotonic solution comprises an isotonic agent solution. Pharmaceutically acceptable isotonic solutions include, but are not limited to, sodium chloride solution, lactose, and dextrose.

An isotonic agent useful according to the present invention can be any pharmaceutically acceptable isotonic agent or a solution thereof. Common isotonic agents include agents selected from the group consisting of sodium chloride, mannitol, lactose, dextrose (hydrous or anhydrous), sucrose, glycerol, and sorbitol, or a solution of any of the foregoing. In some embodiments, a provided reconstituted formulation comprises an isotonic agent, which is sodium chloride or a solution thereof. In some embodiments, sodium chloride is present in an isotonic amount, such that the final concentration of sodium chloride is about 0.1%, about 0.25%, about 0.65%, or about 0.9%.

(b) Dual-Function Containers

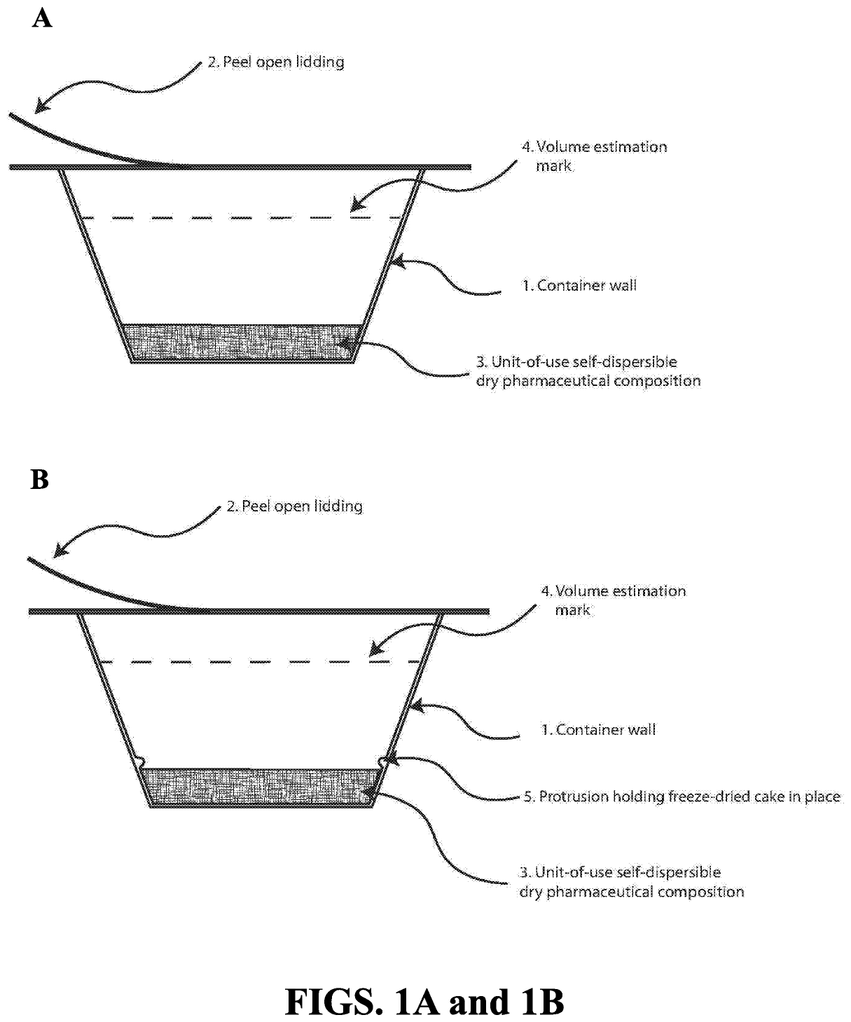

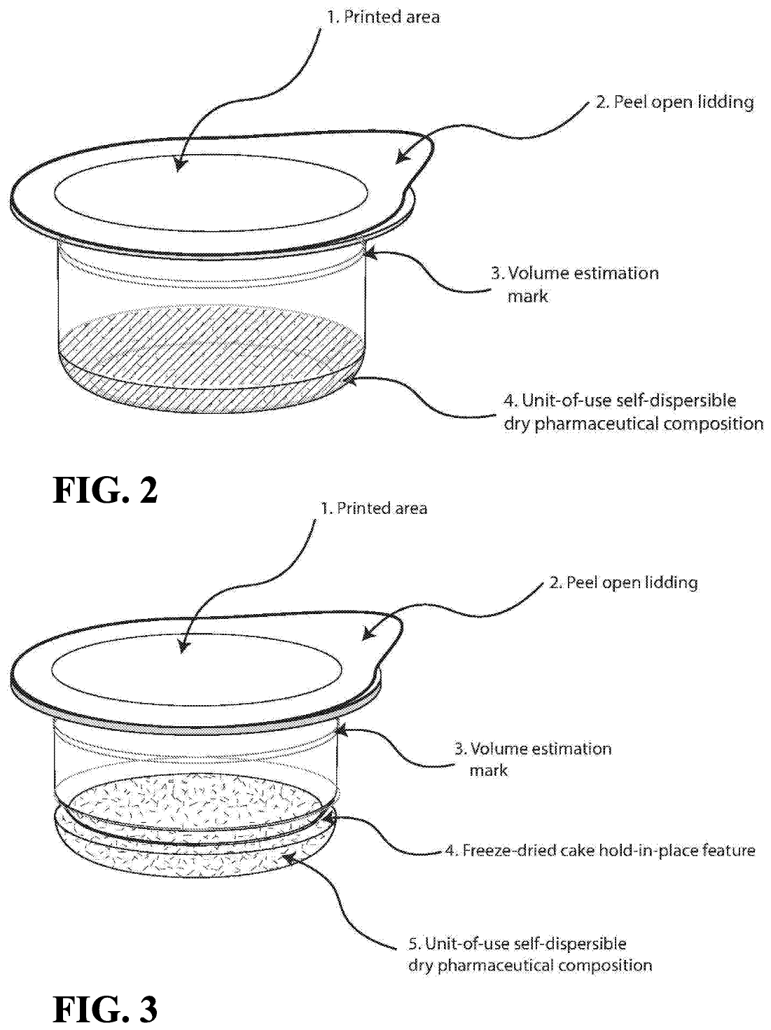

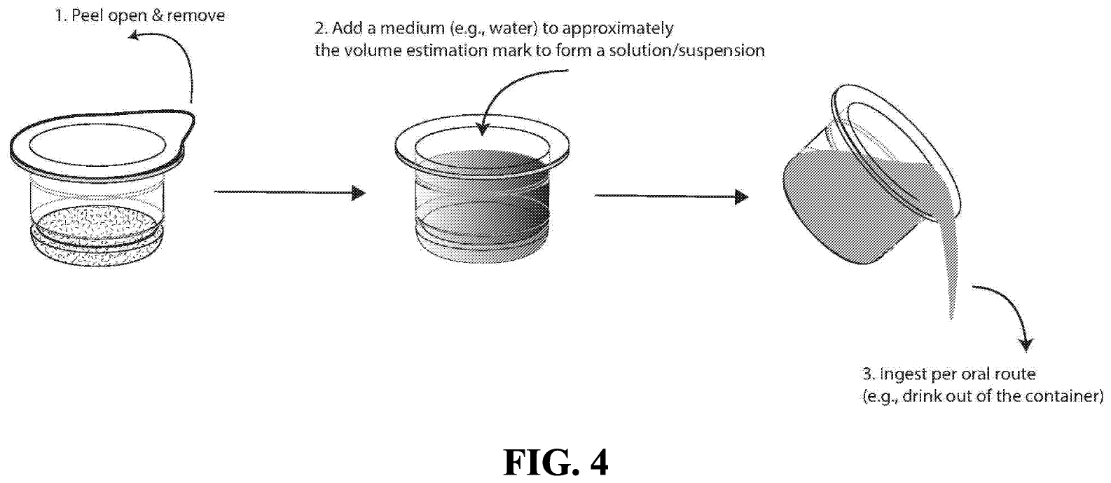

The container of the disclosed unit dose system possesses a dual function as a primary package compartment and a reconstitution device for the pharmaceutical composition, as shown in FIGS. 1-4.

The suitable size of the container should meet at least the following requirements: (1) the volume should be big enough for holding any dry unit-of-use self-dispersing medication formulation; (2) the container should have extra volume to allow reconstitution of the dry unit-of-use self-dispersing medication formulation into liquid oral suspension; and (3) the volume should not have extra space that is unnecessary for meeting requirements one and two and be too bulky for increasing the costs of storage and shipping. For most of the applications, the suitable volume of the unit-of-use container is at least five times greater than the volume of the dry pharmaceutical composition. In some embodiments, the volume of the unit-of-use container, for example, the interior volume defined by the container, is about or less than 50 mL. In some embodiments, the volume of the unit-of-use container ranges from about 5 mL to 50 mL, e.g., from about 10 mL to 30 mL.

The container can be made of various materials, including plastics or glass, or any suitable materials. The materials should be are safe for holding human ingestible medications, protect medication composition stability, do not leach unacceptable level of harmful substance, and can be processed into a container shape.

Suitable plastics materials may include any thermosoftening or thermosetting plastics that can be made into a container shape. Examples of these plastics include without limitation polyethylene (PE) including high-density polyethylene (HDPE) and low-density polyethylene (LDPE), polyvinyl chloride (PVC), polypropylene (PP), nylon, polyterpthalate (PET), polycarbonate, cyclic olefin copolymers (COC), and polystyrene. These plastics can be laminated with each other in duplex or triplex form to provide properties that can not be possessed by individual plastic. Physical properties such as gas and moisture barrier protection, chemical compatibility, leaching, and sorption of individual plastics can be modified by combination use of these plastics through lamination or coextrusion. Additives can also be added to the plastics. Common additives are light blockers, pigments, antistatic agents, etc., that can be added to the plastics to provide additional advantageous properties.

Glass is the most inert and impermeable material. Three main categories of pharmaceutical glass have been established in USP and EP 3.2.1: Type I, II, and III glass. Type I glass is borosilicate based and is the least reactive glass composition available. Type II and Type III glass are soda-lime based on Type II being less reactive than type III but slightly more reactive then Type I. Type II glass is commonly used with aqueous products that will remain below pH 7 for the duration of the intended shelf life. Type III glass is routinely used for packaging dry powders or liquid products that are not sensitive toward alkali. The colored glass, such as amber glass, can be produced by using additives such as nickel, sulfur, or carbon to provide light protection. All three types of glass are suitable for forming the containers in this invention.

In some embodiments, the body of the container is constructed highly rigid for holding a freeze-dried cake. Such rigid container construction can be produced by using rigid plastics or glass or rigid plastic construction. The rigidness of the container should be so that the deformation of the container will not exert an external force that can cause the freeze-dried cake to crumble.

In some embodiments, as shown in FIGS. 1B and 3, the rigid container can further contain a “mechanism” that can hold a freeze-dried cake at the bottom of the container. The mechanism can be in the form of a partial or full ring of protrusion on the wall toward inside of the container to prevent an in situ formed freeze-dried cake to slip out of place and break loose during storage, shipping, and use. A loose freeze-dried cake can easily break into loose powders that produce dust in the container when agitated. The rigid body container in combination with the “mechanism” for a freeze-dried cake can create a freeze-dried pharmaceutical composition that is powder-free. The height of the extrusion ring can be adjusted according to the thickness of the freeze-dried cake to keep it always above the cake to be functional. In some embodiments, the container is a rigid body container. In some embodiments, the container comprises an inner protrusion allowing the pharmaceutical composition to be held in place. The inner protrusion may be a circumferential protrusion and can be part of a recessed ring positioned above the bottom of the container.

There are circumstances that such a design feature can provide important benefits. For example, cytotoxic oncology drugs are toxic. If not handled properly, they can cause harm to unintended individuals such as doctors, nurses, parents, or other caregivers who handle such medications. Such harm can be particularly prominent when the medication is in powder form. It is currently a common practice for compounding dry powder to a liquid suspension that the container is tapped to loosen the powder before opening container to add reconstitution media. Such powdery forms produce drug dust that can float in the air once the container is opened. The dust can be inhaled by the person opening the container and cause unnecessary harm. Another example is when the medication is a narrow therapeutic index drug. The same dusting problem can cause potency loss due to drug dust floats into the air when the container is tapped and opened or sticks to the wall and cover seal, thus not evading from being reconstituted into the unit-of-use liquid suspension.

(c) Removable Seal Cover

In some embodiments, a removable seal cover, such as a peel-open lidding, can be applied to the container opening, sealing the container with its content in it. The peelable lidding seals the dry pharmaceutical composition inside the plastic or glass container to preserve the self-dispersing property of the dry composition. The seal cover can be made of aluminum foil or aluminum foil laminates (e.g., aluminum/plastic or aluminum/paper/plastic) that have low permeability for moisture and air. The sealing can be applied by an adhesive or by means such as heat induction. The sealed container protects the dry pharmaceutical composition from moisture, light, or oxygen, etc., and provides stability of the actives in the dry pharmaceutical composition to assure sufficient shelf-life. The lidding can be peel-opened to allow the dry pharmaceutical composition in the container to be constituted into suspension. The surface of the seal can include the necessary information to identify the drug product. The sealed containers can include a bar code printed thereon to meet the serialization requirements by regulatory agencies.

In some embodiments, the peel-open lidding may have tamper-evident features, which according to the regulations of the Food and Drug Administration (21 C.F.R. 211.132), is one having one or more indicators or barriers to entry which, if breached or missing, can reasonably be expected to provide visible evidence to consumers that tampering has occurred. The peel-open lidding may further have child-resistant and/or senior-friendly packaging features that meet requirements outlined in Code of Federal Regulations (16 C.F.R. 1700.15 (b) (1)), 16 C.F.R. 1700.15 (b)(2)(iii), 16 C.F.R. 1700.20 (a)(3)(i), and 16 C.F.R. 1700.20 (a) (3) (iv)). The peel-openlidding may further covered by a cap having child-resistant and/or senior-friendly packaging features that meet requirements outlined in Code of Federal Regulations (16 C.F.R. 1700.15 (b) (1)), 16 C.F.R. 1700.15 (b)(2)(iii), 16 C.F.R. 1700.20 (a)(3)(i), and 16 C.F.R. 1700.20 (a) (3) (iv)).

(d) Method for Forming Unit Dose Systems

(1) Containers

The containers can be produced by processes such as thermoforming, injection molding, or blow molding.

Thermoforming is a manufacturing process where a plastic sheet is heated to a pliable forming temperature, formed to a specific shape in a mold, and trimmed to create a usable product. The plastic sheet is fed from a roll and transported through an oven for heating to forming temperature. The heated sheet then indexes into a form station where a container mold and pressure-box close on the sheet, with vacuum applied to remove trapped air and to pull the material into or onto the mold along with pressurized air to form the plastic to the detailed shape of the container mold. Then, a burst of reverse air pressure is actuated from the vacuum side of the mold as the form tooling opens, commonly referred to as air-eject, to break the vacuum and assist the formed parts off of, or out of, the mold. A stripper plate may also be utilized on the mold as it opens for ejection of more detailed parts or those with negative-draft, undercut areas. The sheet containing the formed parts then indexes into a trim station on the same machine, where a die cuts the parts from the remaining sheet web or indexes into a separate trim press where the formed parts are trimmed into containers.

Injection molding is a manufacturing process for producing parts by injecting molten plastics or glass into a mold. Typically pelletized plastics or glass for the containers are fed into a heated barrel, mixed, and injected by high pressure into a mold cavity, where it cools and hardens to the configuration of the cavity. Once the required cool temperature has been achieved, the mold opens to eject the molded containers.

Blow molding is a manufacturing process by which hollow plastic or glass parts are formed and can be joined together. In general, there are three main types of blow molding: extrusion blow molding, injection blow molding, and injection stretch blow molding. The blow molding process begins with melting down the plastic or glass and forming it into a parison or a preform, which is a tube-like piece of plastic with a hole in one end through which compressed air can pass. The parison or preform is then clamped into a mold, and the air is blown into it. The air pressure then pushes the plastic or glass out to match the mold. Once the plastic or glass has cooled and hardened the mold opens up, and the part is ejected.

(2) Processes to Produce Dry Powder Blend, Freeze-Dried Dry Powder Blend, and Freeze-Dried Cake

Contained in the container is a self-dispersible dry pharmaceutical composition. The self-dispersible dry pharmaceutical composition can be in different forms and produced by various methods. In one embodiment, the self-dispersible dry pharmaceutical composition is a powder blend. The powder blend can be produced by any conventional processes for oral solid doses. For example, a powder blend can be produced by a direct mixing process in which active pharmaceutical ingredient(s) is mixed with suitable excipients. A powder blend can also be produced by granulation processes such as dry granulation process by roller compaction or slugging, or by wet granulation process by either high/low shear granulators or fluid bed. The powder blend contains excipients that enable or facilitate the blend to be self-dispersing when in contact with a liquid medium. Such excipients can be selected from groups of surfactants, lipids, highly water-soluble materials such as sugars, and disintegrants. A measured weight of the self-dispersing powder blend containing a unit-of-use dose of active is then filled into the container and sealed with the peel-open lidding.

In another embodiment, the dry pharmaceutical composition can be a freeze-dried powder or cake inside the container. The active pharmaceutical ingredients can be dissolved in a suitable medium to form a solution. The active pharmaceutical ingredients, when not soluble, can be dispersed in a suitable medium to form a suspension. The active pharmaceutical ingredients with unpalatable taste can be complexed with a suitable ion exchange resin to conceal their unpleasant tastes. The drug-ion exchange resin complex can be dispersed in a suitable medium to form a suspension. Additional inactive ingredients, such as preservatives, sweeteners, coloring agents, and flavoring agents, antioxidants, pH adjusting agents, can be added to the solution or suspension. An aliquot of the resulted solution or suspension that contains a unit-of-use dose of active can be filled into the containers. The filled containers can be placed into a freeze dryer, and the filled solution or suspension can be dried into dry powder or cake. The containers containing the freeze-dried powder or cake will then be sealed with a seal cover, e.g., a peel-open lidding. Alternatively, the solution or suspension can be spread onto trays that can be dried into cakes in a freeze dryer. The freeze-dried cakes will then be broken into powder or granules by proper milling step. The powder or granules that contains a single therapeutic dose can be filled into the container and sealed with the peel-open lidding.

(3) Processes to Produce Lidding and Sealing

The container containing the dry pharmaceutical composition is closed by a lidding foil commonly used in pharmaceutical packaging for blister or bottle sealing. A typical lidding foil is 25 μm aluminum foil coated with a heat-sealing polymer on the inside and a print primer on the outside. The lidding foil can be bond to the container through an induction sealing process in which the aluminum foil is heated by eddy currents generated by an electromagnetic field, and the heat causes the heat-seal polymer to melt and bond to the lip of the container. The print primer coated outside of the lidding foil can be pre-printed with product information such as product name, strength, expiration date, and bar code, etc. The sealed containers can be further packaged into a kit or pack for a course of treatment or a treatment period.

### B. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The expression “dosage preparation” refers to the form or context in which a formulation is stored and/or used prior to or during administration to a subject. For example, a “dosage preparation” containing a formulation may constitute or comprise the formulation in the context of a vial appropriate for storage and/or administration. A dosage preparation may constitute or comprise a formulation in the context of a container that protects the formulation from light (e.g., UV light). Alternatively, a dosage preparation may constitute or comprise a formulation in the context of a container which does not protect the formulation from exposure to light.

The term, “dry powder formulation” or “dry powder composition” refers to a dry, solid composition, and encompasses dried compositions prepared by freeze-drying (e.g., lyophilization) or other appropriate methods (e.g., spray drying, supercritical fluid formation, etc.) to achieve production of a dried amorphous cake form. Lyophilization is a process of freeze-drying in which water is sublimed from the product after it is frozen, optionally by applying a vacuum. Specifics of lyophilizing or freeze-drying are known in the art and described, for example, in Remington's Pharmaceutical Sciences, Chapter 84, page 1565, 18^{th} Edition, A. R. Gennaro, Editor, 1990, Mack Publishing Company. Techniques other than lyophilization which may also be used for preparation of dry powder formulation(s) (e.g., dried samples), and particularly for preparation of amorphous dry powder formulations, are known in the art, include, but are not limited to, sterile powder filling of the components, singly, or as a complete blend, spray drying, tray drying, sizing processes including milling and/or screening and precipitation. In certain embodiments, inventive dry powder formulations are in the form of a cake (e.g., an amorphous cake).

As used herein, an “effective amount” of a compound or pharmaceutically acceptable formulation can achieve a desired therapeutic and/or prophylactic effect. In some embodiments, an “effective amount” is at least a minimal amount of a compound, or formulation containing a compound, which is sufficient for treating one or more symptoms of a disorder or condition.

The term “formulation,” in general, refers to a preparation that includes at least one pharmaceutically active compound optionally in combination with one or more excipients or other pharmaceutical additives for administration to a subject. In general, particular excipients and/or other pharmaceutical additives are typically selected with the aim of enabling a desired stability, release, distribution, and activity of active compound(s) for applications.

“Therapeutically active agent,” “active agent,” and “active” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition. They include a biologically active substance that is useful for therapy (e.g., human therapy, veterinary therapy), including prophylactic and/or therapeutic treatment. Therapeutically active agents can be organic molecules that are drug compounds, peptides, proteins, carbohydrates, monosaccharides, oligosaccharides, polysaccharides, nucleoprotein, mucoprotein, lipoprotein, synthetic polypeptide or protein, small molecules linked to a protein, glycoprotein, steroid, nucleic acid, DNA, RNA, nucleotide, nucleoside, oligonucleotides, antisense oligonucleotides, lipid, hormone, and vitamin. Alternatively or additionally, therapeutically active agents can be any substance used as a medicine for treatment, prevention, delay, reduction or amelioration of a disease, condition, or disorder. Among therapeutically active agents useful in the formulations of the present invention are opioid antagonist compounds, opioid analgesic compounds, and the like. Further detailed description of agents useful as therapeutically active agents is provided below. The term “therapeutically active agent” can also refer to a first agent that increases the effect or effectiveness of a second agent, for example, by enhancing potency, increasing availability, and/or reducing adverse effects of a second agent.

In general, pharmaceutically acceptable salts include, but are not limited to, chloride, bromide, iodide, nitrate, sulfate, bisulfate, phosphate, acid phosphate, isonicotinate, acetate, lactate, salicylate, citrate, tartrate, pantothenate, bitartrate, carbonate, ascorbate, succinate, maleate, gentisinate, fumarate, gluconate, glucoronate, saccharate, formate, carboxylate, benzoate, glutamate, sulfonate, methanesulfonate, ethanesulfonate, benzensulfonate, p-toluenesulfonate, selenate, and pamoate (i.e., 1, l′-methylene-bis-(2-hydroxy-3-naphthoate)) salts of compounds.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), etc.). Pharmaceutical grade further incorporates suitability for administration by means including topical, ocular, parenteral, nasal, pulmonary tract, mucosal, vaginal, rectal, intravenous, and the like.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “I” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### C. EXAMPLES

Examples are provided below to demonstrate the benefits of the invention for various therapeutic active agents as a unit-of-use, self-dispersing, easy to use, dry suspension dosage form over the current suspension dosage forms. These benefits include but not limited to: (1) a single dose of use without the need for dose division; (2) in addition, the container is used as both the packaging and the reconstitution device. No separate dose division device and reconstitution container are needed; (3) simple and easy reconstitution procedure. It eliminates the need for accurate volume measuring of reconstitution medium (e.g., pharmacopeia grade purified water) and vigorous shaking and other complex preparation steps for ensuring the delivery of an accurate therapeutic dose. Notably, the reconstitution procedure does not require a professional (e.g., a pharmacist) to perform, and the unit does not need to be constituted in a pharmacy or a compounding facility. The reconstitution procedure can be performed by an adult (e.g., a patient) in any normal setting, including household, working, school, office, and travel. Even in hospital and pharmacy settings, the simple and easy reconstitution procedure will save time and reducing potential errors for healthcare providers.

The unit-of-use package of dry suspension of an active will have a longer shelf-life (e.g., 24 months or longer) compared to a liquid suspension of the same active (e.g., drug), since the package will not be constituted until the time of administration. This constitutes a significant improvement from the short “in use” shelf-life (e.g., 14-60 days) of a liquid suspension. Further, it also eliminates the need for the “in use” refrigeration storage condition for constituted solution or suspension. This is particularly beneficial for on-the-go traveling users.

### Example 1

Unit-of-Use, Self-Dispersing Dry Suspension of a Cytotoxic Drug.

The following dry suspension composition containing a retinoid oncology drug Bexarotene composition was prepared according to the composition in the table below.

| Ingredients | % By Weight |
|---|---|
| Bexarotene, USP (micronized) | 3.48% |
| Poloxamer 188 | 0.89% |
| Sodium Alginate CR8223 | 0.09% |
| Mannitol | 2.68% |
| Purified water* | 92.86% |

*Removed during processing

The liquid suspension, 2 mL, was filled into a 23 mL glass container and freeze-dried using a Labconco freeze dryer (FreeZone 2.5) operating at a 0.11 mbar vacuum pressure and −50° C. for 20 hours, to form the dry suspension. A dust-free freeze-dried cake of Bexarotene was formed. The dry suspension was either capped or sealed until the water reconstitution evaluation.

### Example 2

Unit-of-Use, Self-Dispersing Dry Suspension of a Hormone Therapy Drug.

The following dry suspension composition containing an inhibitor of CYP17 (17α-hydroxylase/C17,20-lyase) Abiraterone acetate was prepared according to the composition in the table below.

| Ingredients | % By Weight |
|---|---|
| Abiraterone Acetate, USP (Micronized) | 2.35% |
| Poloxamer 188 | 0.90% |
| Sodium Alginate CR8223 | 0.09% |
| Mannitol | 2.71% |
| Purified water* | 93.95% |

*Removed during processing

The liquid suspension, 5 mL, was filled into a 23 mL glass container and freeze-dried using a Labconco freeze dryer (FreeZone 2.5) operating at 0.11 mbar vacuum pressure and −50° C. for 24 hours, to form the dry suspension. A dust-free freeze-dried cake of Abiraterone Acetate was formed. The dry suspension was either capped or sealed until the water reconstitution evaluation.

### Example 3

Unit-of-Use, Self-Dispersing Dry Suspension of a Synthetic Nucleoside Analogue Drug.

The following dry suspension composition containing a synthetic nucleoside analogue Acyclovir was prepared according to the composition in the table below.

| Ingredients | % By Weight |
|---|---|
| Acyclovir, USP (Micronized) | 3.71% |
| Poloxamer 188 | 0.89% |
| Sodium Alginate CR8223 | 0.09% |
| Mannitol | 2.67% |
| Purified water* | 92.65% |

*Removed during processing

The liquid suspension, 5 mL, was filled into a 23 mL glass container and freeze-dried using a Labconco freeze dryer (FreeZone 2.5) operating at a 0.11 mbar vacuum pressure and −50° C. for 20 hours, to form the dry suspension. The dry suspension was either capped or sealed until the water reconstitution evaluation.

### Example 4

Unit-of-Use, Self-Dispersing Dry Suspension of a Calcium Channel Blocker.

The following dry suspension composition containing a hypertension drug Felodipine was prepared according to the composition in the table below.

| Ingredients | % By Weight |
|---|---|
| Felodipine, USP (micronized) | 0.19% |
| Poloxamer 188 | 0.92% |
| Sodium Alginate CR8223 | 0.09% |
| Mannitol | 2.77% |
| Purified water* | 96.03% |

*Removed during processing

The liquid suspension, 5 mL, was filled into a 23 mL glass container and freeze-dried using a Labconco freeze dryer (FreeZone 2.5) operating at 0.11 mbar vacuum pressure and −50° C. for 24 hours, to form the dry suspension. The dry suspension was either capped or sealed until the water reconstitution evaluation.

### Example 5

Unit-of-Use, Self-Dispersing Dry Suspension of a Central alpha2A-Adrenergic Receptor Agonist and Containing Ion-Exchange Resin for Bitter Taste Concealing.

The following dry suspension composition containing a central alpha-2A-adrenergic receptor agonist Guanfacine was prepared according to the composition in the table below. The Guanfacine HCl active was first complexed with an ion-exchange resin Amberlite IRP64 for concealing its bitter taste purpose. The formed ion-exchange resin-active complex was then suspended in the aqueous solution containing Poloxamer 188, sodium alginate, and mannitol to form a suspension.

| Ingredients | % By Weight |
|---|---|
| Guanfacine HCl, USP | 0.80% |
| Amberlite IRP64 | 3.67% |
| Poloxamer 188 | 0.92% |
| Sodium Alginate CR8223 | 0.09% |
| Mannitol | 2.75% |
| Purified water* | 91.78% |

*Removed during processing

The liquid suspension, 0.5 mL, was filled into a 9 mL plastic container and freeze-dried using a Labconco freeze dryer (FreeZone 2.5) operating at a 0.2 mbar vacuum pressure and −50° C. for 24 hours, to form the dry suspension.

The dry suspensions were either capped or sealed until the water reconstitution evaluation.

### Example 6

Unit-of-Use, Self-Dispersing Dry Suspension of Nifedipine.

The following dry suspension composition containing an antianginal drug Nifedipine was prepared according to the composition in the table below.

| Ingredients | % By Weight |
|---|---|
| Nifedipine, USP | 0.95% |
| Poloxamer 188 | 0.92% |
| Sodium Alginate CR8223 | 0.09% |
| Mannitol | 2.75% |
| Purified water | 95.29% |

*Removed during processing

The liquid suspension, 5 mL, was filled into a 23 mL glass container and freeze-dried using a Labconco freeze dryer (FreeZone 2.5) operating at a 0.11 mbar vacuum pressure and −50° C. for 20 hours, to form the dry suspension. The dry suspension was either capped or sealed until the water reconstitution evaluation.

### Example 7

Unit-of-Use, Self-Dispersing Dry Suspension of an Antiviral Drug and Containing Ion-Exchange Resin for Bitter Taste Concealing.

The following dry suspension composition containing an antiviral drug Oseltamivir was prepared according to the composition in the table below. The Oseltamivir Phosphate active was first complexed with the ion-exchange resin Amberlite IRP69 for concealing the bitter taste of Oseltamivir Phosphate. The formed ion-exchange resin-active complex was then suspended in the aqueous solution containing Poloxamer 188, sodium alginate, and mannitol to form a liquid suspension.

| Ingredients | % By Weight |
|---|---|
| Oseltamivir phosphate, USP | 0.44% |
| Amberlite IRP69 | 3.67% |
| Poloxamer 188 | 0.92% |
| Sodium Alginate CR8223 | 0.09% |
| Mannitol | 2.76% |
| Purified water* | 92.12% |

*Removed during processing.

The liquid suspension, 1.3 mL, was filled into a 9 mL plastic container and freeze-dried using a Labconco freeze dryer (FreeZone 2.5) operating at 0.2 mbar vacuum pressure and −50° C. for 24 hours, to form the dry suspension.

The dry suspensions were either capped or sealed until the water reconstitution evaluation.

### Example 8

Unit-of-Use, Self-Dispersing Dry Solution of a Calcium Channel Blocker.

The following dry solution composition containing a hypertension drug Amlodipine Besylate was prepared according to the composition in the table below.

| Ingredients | % By Weight |
|---|---|
| Amlodipine Besylate, USP | 0.27% |
| Mannitol | 1.01% |
| Sucralose | 0.04% |
| Natural mango flavor solution (0.1%) | 0.002% |
| Purified water* | 98.67% |

*Removed during processing

The liquid suspension, 2.5 mL, was filled into a 23 mL glass container and freeze-dried using a Labconco freeze dryer (FreeZone 2.5) operating at 0.22˜0.25 mbar vacuum pressure and −49° C. for 21 hours, to form the dry solution. The dry solution was either capped or sealed until the water reconstitution evaluation.

### Example 9

Water Reconstitution Evaluation of the Unit-of-Use, Self-Dispersing Dry Suspensions Prepared According to the Invention.

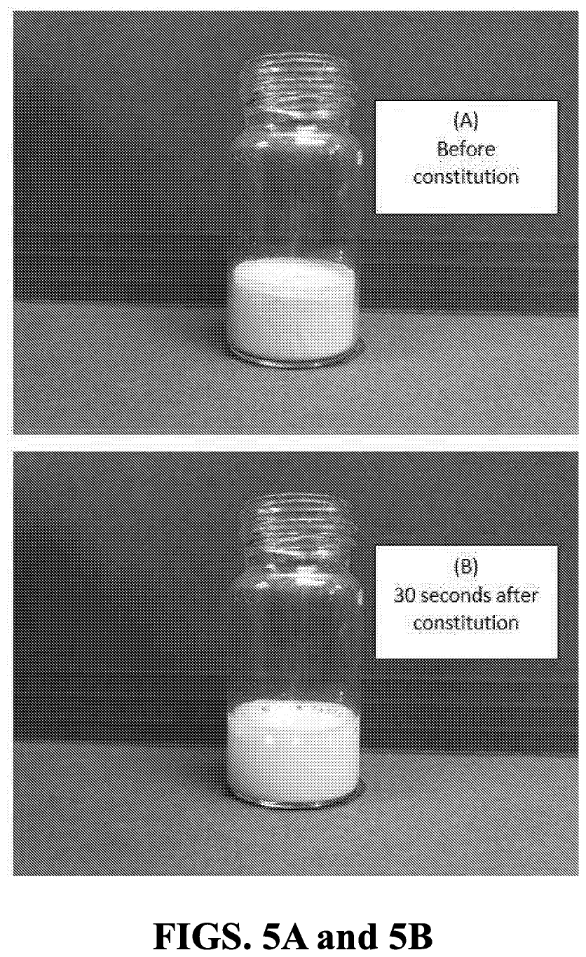

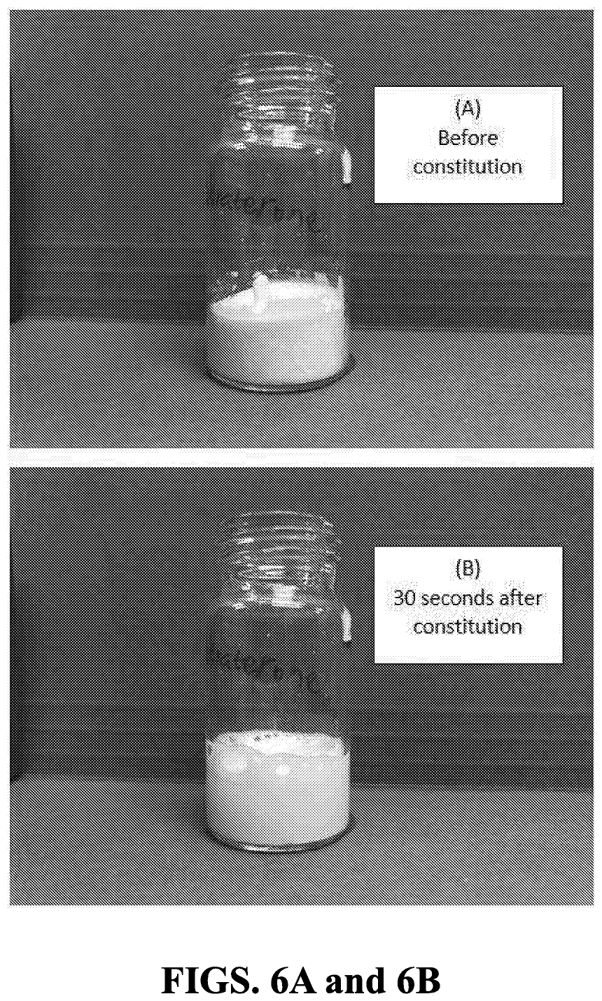

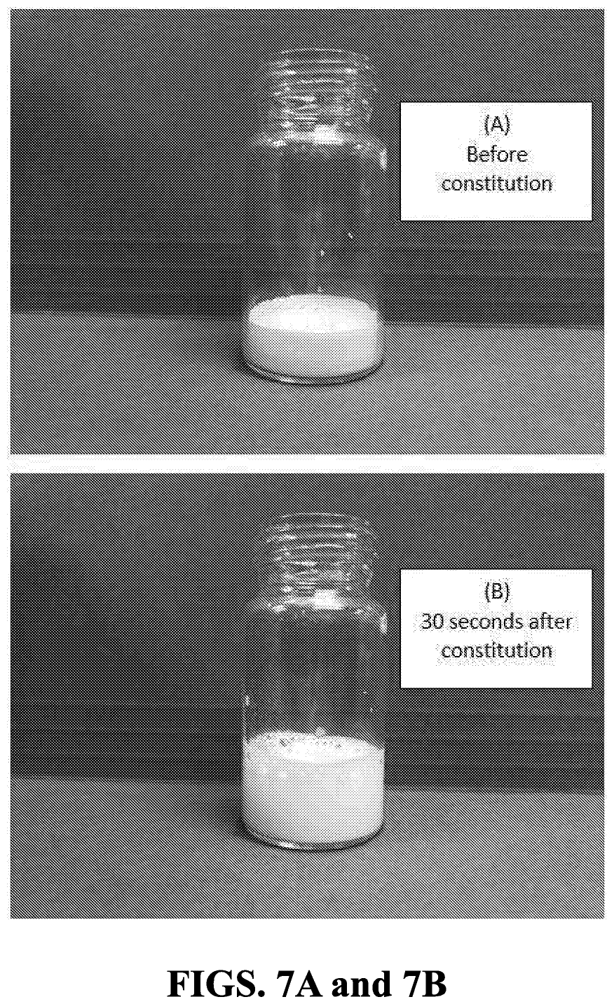

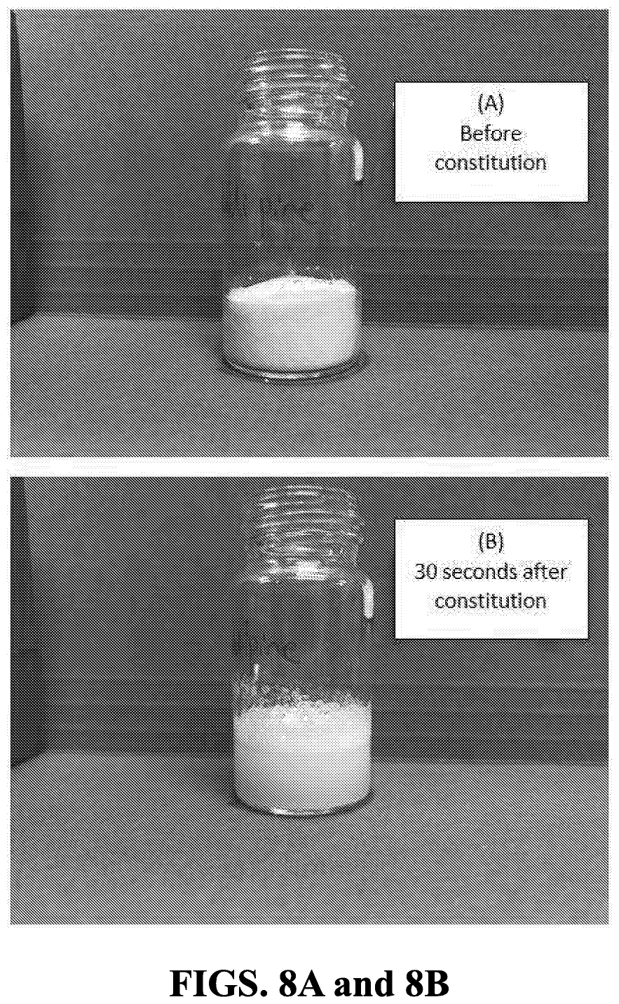

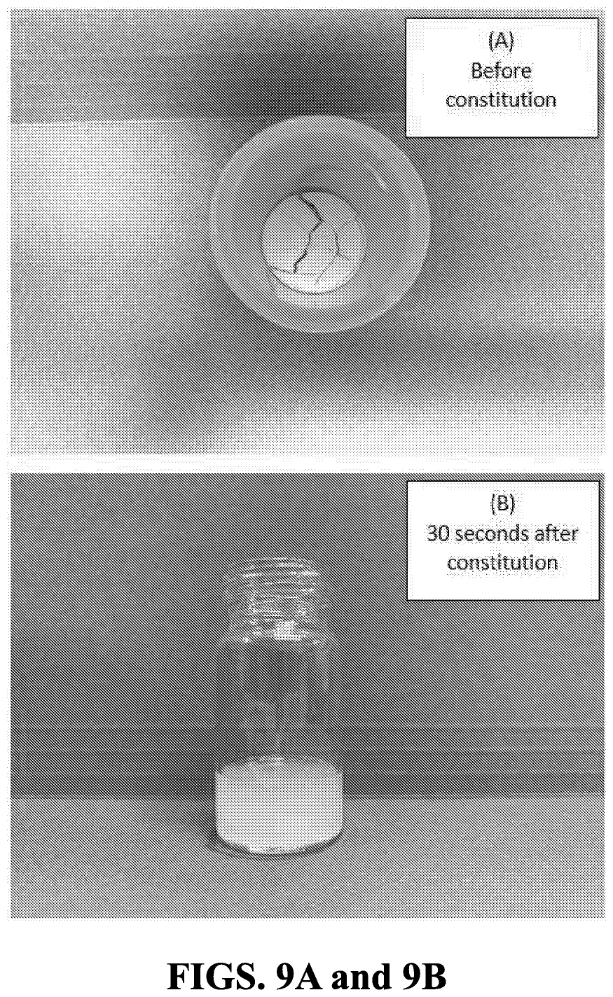

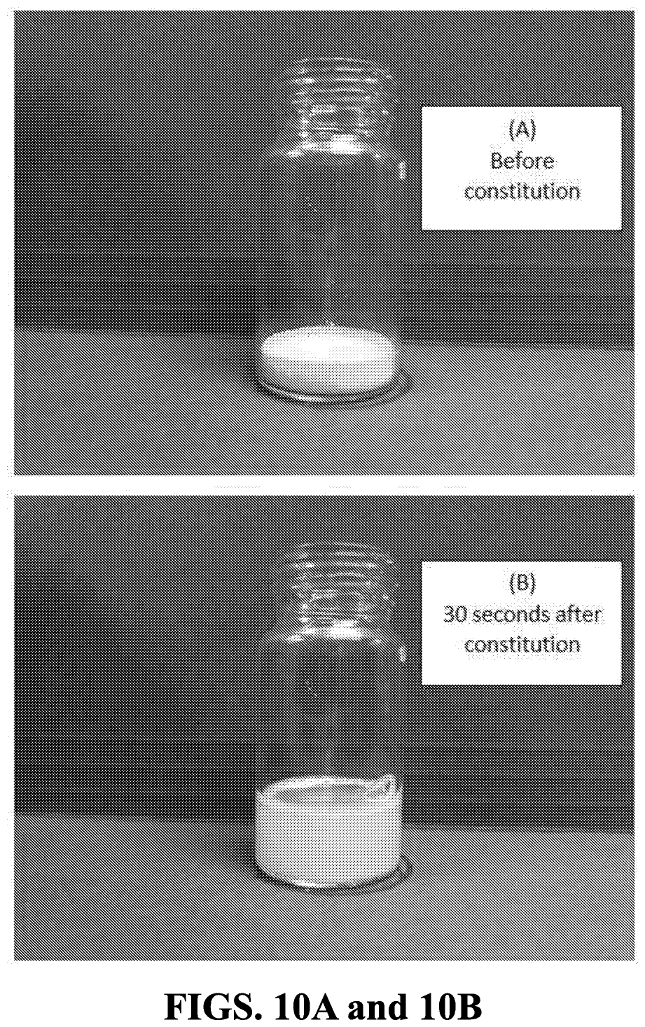

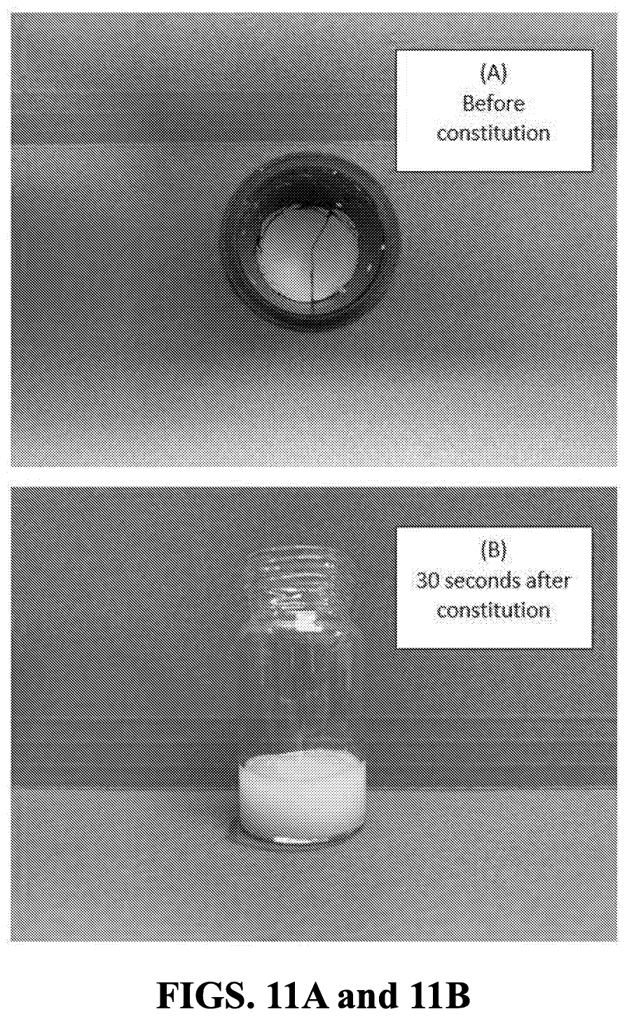

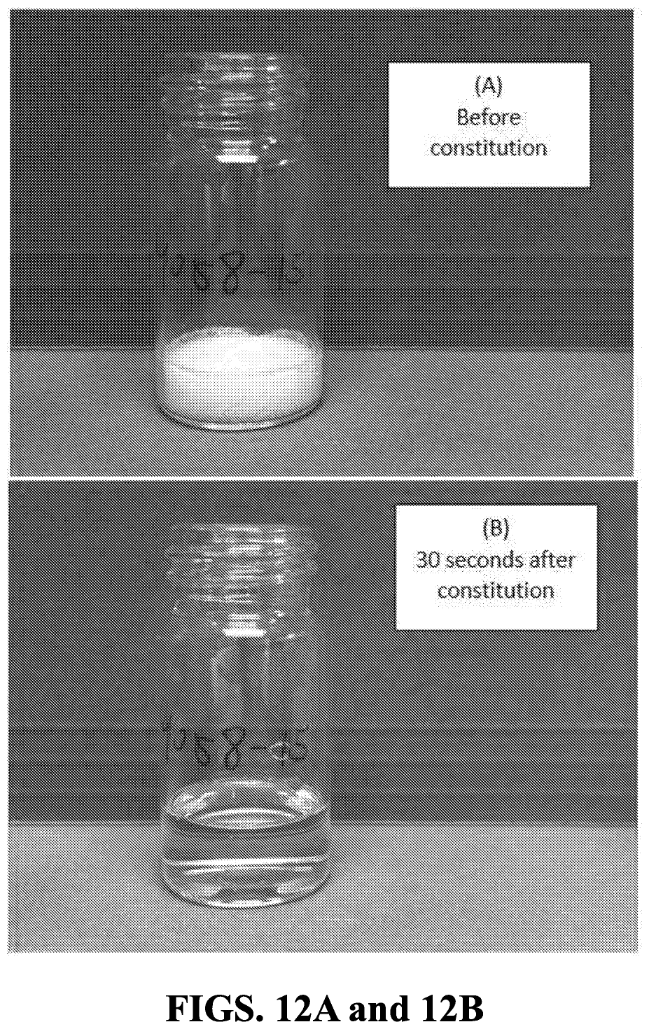

The freeze-dried samples prepared in Examples 1-7 were evaluated for in-the-container reconstitution with tap water. An aliquot of 5 mL tap water was measured and placed in a test tube. This water was poured into the plastic or glass container to constitute the freeze-dried samples prepared in Examples 1-7 into a suspension. Before the water reconstitution step, a photo was taken. A timer was used to record the elapsed time of the reconstitution. At 30-second elapsed time point, the appearance of constituted suspension was observed, and a photo was taken. The photos are provided in FIGS. 5-12. For constituted suspension in the plastic container, it was promptly transferred to a glass container at the 30-second elapsed time point for the observation and photo-taken purposes.

The freeze-dried compositions were quickly self-dispersed in the 5 mL water and formed a suspension as shown in the photos within 30 seconds without the need for shaking. The reconstitution was simple and easy, without the need for a separate container. The constituted Oseltamivir suspension is suitable for oral administration directly out of the plastic or glass container.

What is claimed is:

1. A pharmaceutical unit dose system for oral administration, comprising:
a self-dispersible dry pharmaceutical composition for oral administration, wherein the pharmaceutical composition comprises at least one active agent in the amount of a single administrative dose and at least one pharmaceutically acceptable excipient that allows the dry pharmaceutical composition to self-disperse in the presence of a liquid medium;
a container for packaging and reconstituting the dry pharmaceutical composition, wherein the container has a volume at least five-fold greater than the volume of the dry pharmaceutical composition; and
a seal cover removably secured to the container by a sealing contact with an opening of the container.

2. The unit dose system of claim 1, wherein the pharmaceutical composition further comprises a preservative, a coloring agent, an antioxidant, a pH adjusting agent, an anti-foaming agent, a suspending agent, a thickening agent, and a combination thereof.

3. The unit dose system of claim 1, wherein the excipient comprises a highly water-soluble material, a disintegrant, or a combination thereof.

4. The unit dose system of claim 1, wherein the excipient comprises a taste-masking agent.

5. The unit dose system of claim 1, wherein the excipient comprises a matrix-forming agent.

6. The unit dose system of claim 1, wherein the dry pharmaceutical composition is in the form of powder blend.

7. The unit dose system of claim 6, wherein the powder blend comprises formulated granules produced from a dry or wet granulation process.

8. The unit dose system of claim 1, wherein the dry pharmaceutical composition is a freeze-dried pharmaceutical composition.

9. The unit dose system of claim 8, wherein the freeze-dried pharmaceutical composition is prepared by in situ freeze-drying a liquid suspension or solution of the pharmaceutical composition in the container.

10. The unit dose system of claim 1, wherein the dry pharmaceutical composition is in the form of a freeze-dried cake.

11. The unit dose system of claim 1, wherein the dry pharmaceutical composition comprises an ion-exchange resin that complex with the active agent to conceal the taste of the active agent.

12. The unit dose system of claim 11, wherein the ion-exchange resin is micronized.

13. The unit dose system of claim 12, wherein the ion-exchange resin has a particle size less than 50 μm.

14. The unit dose system of claim 11, wherein the ion-exchange resin is Amberlite IRP-69, Amberlite IRP-64, Colestipol hydrochloride, or Duolite AP143/1093.

15. The unit dose system of claim 1, wherein the active agent is selected from the group consisting of analeptic agents; analgesic agents; anesthetic agents; antiasthmatic agents; antiarthritic agents; anti-cancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyper activity disorder active agents; cardiovascular agents including calcium channel blockers and antianginal agents; central nervous system (CNS) agents; beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide active agents; psychoStimulants; sedatives; Sialagogues, steroids; Smoking cessation agents; Sympathomimetics; tranquilizers; vasodilators; beta-agonist; tocolytic agents; and combinations thereof.

16. The unit dose system of claim 1, wherein the active agent is a cytotoxic anti-cancer or anti-tumor agent, an antiviral, or an anti-bacterial agent.

17. The unit dose system of claim 1, wherein the liquid medium is water.

18. The unit dose system of claim 1, wherein the container has a dual function as a primary package compartment and a reconstitution device for the pharmaceutical composition.

19. The unit dose system of claim 1, wherein the container is cylindrical or substantially cylindrical.

20. The unit dose system of claim 1, wherein the container comprises a volume marking indicating a volume for reconstitution of the pharmaceutical composition.

21. The unit dose system of claim 20, wherein the volume for reconstitution of the pharmaceutical composition indicated by the volume marking is about or less than 80% of the interior volume defined by the container.

22. The unit dose system of claim 21, the interior volume of the container is between about 5 mL and about 50 mL.

23. The unit dose system of claim 21, the interior volume of the container is between about 10 mL and about 30 mL.

24. The unit dose system of claim 1, wherein the container is formed of a plastic selected from the group consisting of polyethylene (PE) including high-density polyethylene (HDPE) and low-density polyethylene (LDPE), polyvinyl chloride (PVC), polypropylene (PP), nylon, polyterpthalate (PET), polycarbonate, cyclic olefin copolymers (COC), polystyrene, and a combination thereof.

25. The unit dose system of claim 1, wherein the container is formed of glass.

26. The unit dose system of claim 1, wherein the container is a rigid body container.

27. The unit dose system of claim 1, wherein the container comprises an inner protrusion allowing the pharmaceutical composition to be held in place.

28. The unit dose system of claim 27, wherein the inner protrusion is a circumferential protrusion.

29. The unit dose system of claim 27, wherein the inner protrusion is part of a recessed ring positioned above the bottom of the container.

30. The unit dose system of claim 1, wherein the seal cover comprises product information.

31. The unit dose system of claim 30, wherein the product information comprises a product name, dosage, ingredients, lot number, expiration date, company information, contact information, serialization barcode, warning message, instruction for use, or a combination thereof.

32. The unit dose system of claim 1, wherein the seal cover is formed of aluminum, paper, plastic, or a combination thereof.

33. The unit dose system of claim 1, wherein the seal cover is an aluminum foil or a plastic film.

34. The unit dose system of claim 1, wherein the seal contact to secure the seal cover to the container is provided by an adhesive.

35. A kit comprising:
the unit dose system of claim 1;
a second container comprising an aqueous carrier; and
optionally an instruction for reconstituting the pharmaceutical composition using aqueous carrier.

36. The kit of claim 35, wherein the aqueous carrier comprises water.

37. The kit of claim 36, wherein the water is purified water, sweetened water, or flavored water.

38. A method for preparing a single unit dose of a dry pharmaceutical composition for oral administration, comprising:
providing the pharmaceutical unit dose system of claim 1;
detaching the seal cover, partially or completely, from the opening of the container; and
adding a reconstitution liquid medium into the container to approximately the volume marking and allowing the dry pharmaceutical composition to self-disperse to form an oral solution or suspension.

39. The method of claim 38, wherein the single unit dose of the dry pharmaceutical composition is prepared without the need for vigorous mixing.

40. The method of claim 38, wherein the unit dose system is provided in the kit of claim 35.

41. A method for forming the pharmaceutical unit dose system for oral administration of claim 1, comprising:
forming the container with an opening;
preparing the self-dispersing dry pharmaceutical composition;
packaging a single dose of the dry pharmaceutical composition into the container; and
securing the seal cover to the container by a sealing contact over the opening of the container.

42. The method of claim 41, wherein the self-dispersing dry pharmaceutical composition is provided in the form of a powder blend.

43. The method of claim 41, wherein the self-dispersing dry pharmaceutical composition is provided in the form of a freeze-dried cake.

44. A method for forming the pharmaceutical unit dose system for oral administration of claim 1, comprising:
forming the container with an opening;
preparing a solution or a liquid suspension of the self-dispersing dry pharmaceutical composition;
adding the solution or the liquid suspension of a single dose of the pharmaceutical composition into the container;
freeze-drying the solution or the liquid suspension to form a freeze-dried cake of the pharmaceutical composition; and
securing the seal cover to the container by a sealing contact over the opening of the container.
